(19) United States
(12) Patent Application Publication
(10) Pub. No.: US 2025/0249480 A1
(43) Pub. Date: Aug. 7, 2025

(54) ULTRASONIC TRANSDUCER DEVICE

(71) Applicant: AUO Corporation, Hsinchu City (TW)

(72) Inventors: Zhen Wah Chew, Hsinchu City (TW); Zheng-Han Chen, Hsinchu City (TW); Pin-Hsiang Chiu, Hsinchu City (TW); Tai-Hsiang Huang, Hsinchu City (TW)

(73) Assignee: AUO Corporation, Hsinchu City (TW)

(21) Appl. No.: 19/186,639

(22) Filed: Apr. 23, 2025

Related U.S. Application Data
(63) Continuation of application No. 18/076,348, filed on Dec. 6, 2022.

(30) Foreign Application Priority Data
Aug. 2, 2022 (TW) 111128897

(51) Int. Cl. B06B 1/02; A61B 8/00

(52) U.S. Cl. CPC B06B 1/02; A61B 8/4483

(57) ABSTRACT

An ultrasonic transducer device includes a first electrode, an insulating layer, an oscillating membrane, a second electrode, and a third electrode. The insulating layer is disposed on the first electrode. The oscillating membrane is disposed over the insulating layer. A cavity is between the oscillating membrane and the insulating layer. The second electrode is disposed on the oscillating membrane. The third electrode is disposed in the cavity and has a plurality of first electrode openings overlapping the second electrode. The second electrode and the third electrode are each located at different sides of the oscillating membrane.

### CROSS-REFERENCE TO RELATED APPLICATION

This application is a continuation application of and claims the priority benefit of a prior application Ser. No. 18/076,348, filed on Dec. 6, 2022, which claims the priority benefits of Taiwan application serial no. 111128897, filed on Aug. 2, 2022. The entirety of each of the above-mentioned patent applications is hereby incorporated by reference herein and made a part of this specification.

### BACKGROUND

### Technical Field

The disclosure relates to a transducer device, and more particularly, to an ultrasonic transducer device.

### Description of Related Art

Ultrasound transducer devices are technology that obtains images by emitting and receiving ultrasound, which can be applied to measure distance, such as being installed in a car to provide judgment on driving distance, in daily life, or can be applied to a medical diagnosis to check the physical condition of a patient. Generally, an ultrasonic transducer device includes multiple ultrasonic transducer units. The cell density of the ultrasonic transducer device may affect the bandwidth and output power of the ultrasonic transducer device, which in turn affects the performance of the ultrasonic transducer device. How to improve the cell density of the ultrasonic transducer device is an issue to be overcome at present.

### SUMMARY

The disclosure provides an ultrasonic transducer device with increased cell density, thereby improving the performance of the ultrasonic transducer device.

The ultrasonic transducer device of the disclosure includes a first electrode, an insulating layer, an oscillating membrane, a second electrode, and a third electrode. The insulating layer is disposed on the first electrode. The oscillating membrane is disposed over the insulating layer, and there is a cavity between the oscillating membrane and the insulating layer. The second electrode is disposed on the oscillating membrane. The third electrode is disposed in the cavity and has a plurality of first openings overlapping the second electrode, and the second electrode and the third electrode are each located on different sides of the oscillating membrane.

### BRIEF DESCRIPTION OF THE DRAWING

FIG. 1A is a schematic top view of an ultrasonic transducer device according to an embodiment of the disclosure.

FIG. 1B and FIG. 1C are schematic cross-sectional views of an ultrasonic transducer device of FIG. 1A taken along the section line A-A′.

FIG. 2 is a schematic top view of an ultrasonic transducer device according to another embodiment of the disclosure.

FIG. 3 is a schematic top view of an ultrasonic transducer device according to another embodiment of the disclosure.

FIG. 4 is a schematic top view of an ultrasonic transducer device according to another embodiment of the disclosure.

FIG. 5 is a schematic top view of Comparative example 1.

FIG. 6 is a schematic top view of Comparative example 2.

FIG. 7 is a schematic top view of Comparative example 3.

### DESCRIPTION OF THE EMBODIMENTS

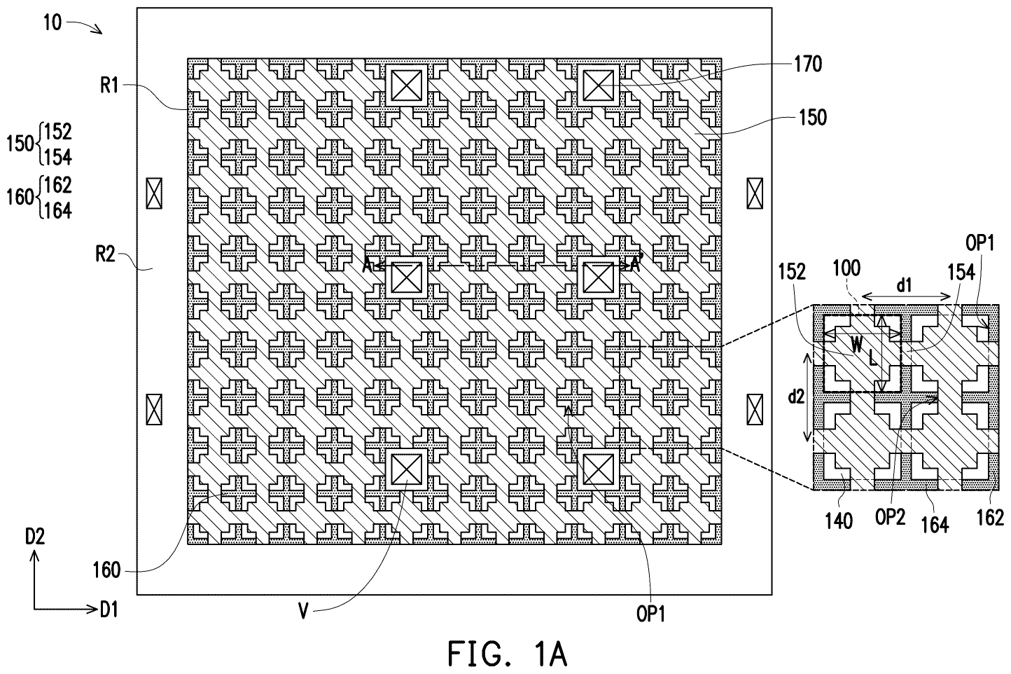

FIG. 1A is a schematic top view of an ultrasonic transducer device according to an embodiment of the disclosure. FIG. 1B and FIG. 1C are schematic cross-sectional views of an ultrasonic transducer device of FIG. 1A taken along the section line A-A′. FIG. 1B is a schematic cross-sectional view of a third electrode 160 in a state where no bias voltage is applied. FIG. 1C is a schematic cross-sectional view of the third electrode 160 in a state where a bias voltage is applied. For clear illustration, an oscillating membrane 140 in FIG. 1A is shown in a perspective manner, and a first electrode 110 and an insulating layer 120 are omitted.

Referring to FIG. 1A and FIG. 1B, an ultrasonic transducer device 10 includes the first electrode 110, the insulating layer 120, the oscillating membrane 140, a second electrode 150, and the third electrode 160.

The materials of the first electrode 110, the second electrode 150, and the third electrode 160 may be titanium (Ti), aluminum (AI), copper (Cu), tungsten (W), molybdenum (Mo), silver (Ag), an alloy thereof, a combination thereof, or other suitable conductive materials. In some embodiments, the first electrode 110, the second electrode 150, and the third electrode 160 may be a single-layer or multi-layer structure (e.g., each is a stacked structure of a titanium layer, an aluminum layer, and a titanium layer). In some embodiments, the materials of the first electrode 110, the second electrode 150, and the third electrode 160 may be the same or different, but the disclosure is not limited thereto. In some embodiments, the entire first electrode 110 may be disposed on the substrate (not shown) without being patterned.

The insulating layer 120 is disposed on the first electrode 110. The material of the insulating layer 120 may be silicon nitride, silicon oxide, silicon oxynitride, aluminum oxide, organic insulating material, or other suitable insulating materials, and the disclosure is not limited thereto. The insulating layer 120 has a top surface 120a and a bottom surface 120b. The bottom surface 120b is located between the top surface 120a and the first electrode 110. In some embodiments, the insulating layer 120 is directly formed on the first electrode 110 and covers the first electrode 110. The area of the insulating layer 120 may be the same as or different from the area of the first electrode 110.

The oscillating membrane 140 is disposed over the insulating layer 120, and there is a cavity 130 between the oscillating membrane 140 and the insulating layer 120. In other words, at least part of the region between the oscillating membrane 140 and the insulating layer 120 is not in direct contact. The oscillating membrane 140 is a thin film, and the material of the oscillating membrane 140 may be silicon nitride, silicon oxide, silicon oxynitride, aluminum oxide, organic insulating material, or other suitable thin film materials. In some embodiments, the oscillating membrane 140 has a first surface 140a and a second surface 140b opposite to the first surface 140a, and the second surface 140b faces the insulating layer 120.

The second electrode 150 and the third electrode 160 are each located on different sides of the oscillating membrane 140. For example, the second electrode 150 is disposed on the first surface 140a of the oscillating membrane 140, and the third electrode 160 is disposed on the second surface 140b of the oscillating membrane 140. That is, the third electrode 160 is disposed in the cavity 130. In some embodiments, the third electrode 160 is a mesh structure. For example, the third electrode 160 includes multiple longitudinal portions 162 extending toward the second direction D2 and arranged along the first direction D1, and multiple transverse portions 164 extending toward the first direction D1 and arranged along the second direction D2, and the first direction D1 and the second direction D2 intersect. In some embodiments, the first direction D1 orthogonally intersects the second direction D2. The third electrode 160 has multiple first openings OP1, and the first openings OP1 are defined by multiple intersected longitudinal portions 162 and transverse portions 164. In the embodiment, the first opening OP1 is square, but the disclosure is not limited thereto. In other embodiments, the first opening OP1 may be rectangular or in other suitable shapes.

The first openings OP1 overlap the second electrode 150. For example, the second electrode 150 may include multiple main parts 152 and multiple connecting parts 154. The area of each main part 152 is greater than the area of each connecting part 154. The main parts 152 are disposed in an array in the first direction D1 and the second direction D2 and overlap the first openings OP1 of the third electrode 160. In some embodiments, the projection of the main part 152 on the insulating layer 120 is square, but the disclosure is not limited thereto. The connecting parts 154 may be connected between adjacent main parts 152 in the first direction D1 and between adjacent main parts 152 in the second direction D2. Accordingly, the connecting parts 154 and the main parts 152 may constitute multiple second openings OP2. In the embodiment, the second opening OP2 is cross-shaped, but the disclosure is not limited thereto. In other embodiments, the second opening OP2 may be rectangular, round, zigzag, or in other suitable shapes.

In some embodiments, the oscillating membrane 140 has multiple through holes V, and the through holes V penetrate through the oscillating membrane 140. The through hole V is an etching hole configured to form the cavity 130 during the fabrication process of the ultrasonic transducer device 10. For example, the method of forming the cavity 130 includes steps as follows. A sacrificial layer (not shown) is formed on the insulating layer 120. Next, the third electrode 160, the oscillating membrane 140 and the second electrode 150 are formed on the sacrificial layer. Through holes V exposing the sacrificial layer are formed on the oscillating membrane 140. Finally, the sacrificial layer is etched through the through holes V to form the cavity 130. After the cavity 130 is formed, a filling material 170 may be filled into the through hole V to close the cavity 130. The filling material 170 is connected to the insulating layer 120. In some embodiments, the filling material 170 includes, for example, cured photoresist, silicon-containing nitride, silicon-containing oxide, or other insulating materials.

Referring to FIG. 1A and FIG. 1C, when the third electrode 160 is applied with a bias voltage (e.g., a DC bias voltage may be applied to the third electrode 160), a voltage difference is generated between the third electrode 160 and the first electrode 110, and the third electrode 160 is brought close to the first electrode 110. After the third electrode 160 is in direct contact with the insulating layer 120, the third electrode 160, the insulating layer 120 and the oscillating membrane 140 form multiple sub-cavities 132, and the sub-cavities 132 are closed spaces and are separated from each other. Accordingly, the third electrode 160, the insulating layer 120, the oscillating membrane 140 and the sub-cavities 132 can constitute multiple ultrasonic transducer units 100 arranged in an array. The ultrasonic transducer units 100 substantially correspond to the first openings OP1 of the third electrode 160, that is, the third electrode 160 may define the dimension of the ultrasonic transducer unit 100. The width W and the length L of the ultrasonic transducer unit 100 are substantially equal to the width and length of the first opening OP1. In the embodiment, the width W of the ultrasonic transducer unit 100 is the same as the length L, and the distance d1 between the adjacent ultrasonic transducer units 100 in the first direction D1 is the same as the distance d2 between the adjacent ultrasonic transducer units 100 in the second direction D2, but the disclosure is not limited thereto. The dimension of the ultrasonic transducer unit 100 and the distances d1 and d2 in the first direction D1 and the second direction D2 may be adjusted according to actual requirements. In the specification, the distance d1 refers to the distance between the centers of two adjacent ultrasonic transducer units in the first direction D1, and the distance d2 refers to the distance between the centers of two adjacent ultrasonic transducer units in the second direction D2. Since the ultrasonic transducer unit 100 is formed by forming a sub-cavity 132 among the insulating layer 120, the oscillating membrane 140 and the third electrode 160 when the third electrode 160 is applied with a bias voltage. Compared to other ultrasonic transducer devices in which the adjacent sub-cavities are filled with materials, in the embodiment, the smaller-sized sub-cavities 132 can be obtained by isolating the sub-cavities 132 through the third electrode 160, thereby improving the cell density of the ultrasonic transducer unit 100.

In some embodiments, the oscillating membrane 140 is wavy when the third electrode 160 is applied with a bias voltage. The crests of the oscillating membrane 140 may correspond to the sub-cavities 132, and the troughs of the oscillating membrane 140 may correspond to the third electrode 160.

In some embodiments, the ultrasonic transducer device 10 may have an active region R1 and a peripheral region R2 located outside the active region R1. The peripheral region R2 may surround the active region R1 or only be located on one or more sides of the active region R1, which is not limited in the disclosure. The ultrasonic transducer unit 100 is located in the active region R1 to sense (e.g., receive or transmit) ultrasonic signals, so the first electrode 110, the second electrode 150 and the third electrode 160 can be located in the active region R1. In some embodiments, some of the through holes V may be located in the peripheral region R2, so that the active region R1 has more space for configuring the ultrasonic transducer units 100, so as to increase the cell density of the ultrasonic transducer device 10. In some embodiments, some of the through holes V may be located in the active region R1, and adjacent through holes V are separated by at least two first openings OP1, that is, at least two ultrasonic transducer units 100 are disposed between adjacent through holes V. Compared to other ultrasonic transducer devices having through holes between adjacent ultrasonic transducer units, the through holes V configured can be reduced in the embodiment, so as to increase the cell density of the ultrasonic transducer device 10. In some embodiments, the through hole V located in the active region R1 corresponds to the first opening OP1 of the third electrode 160.

In some embodiments, after applying a DC bias voltage to the third electrode 160, in the ultrasonic transducer unit 100, an AC bias voltage can be applied to the second electrode 150 so that the oscillating membrane 140 can oscillate back and forth to emit ultrasonic waves.

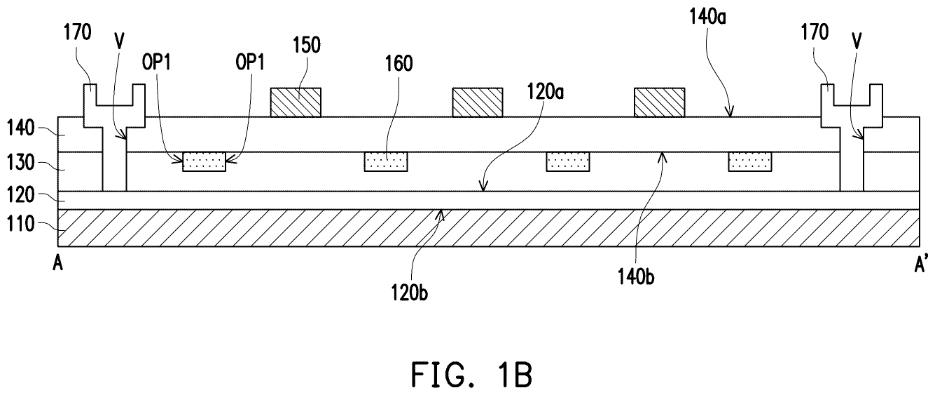

FIG. 2 is a schematic top view of an ultrasonic transducer device according to another embodiment of the disclosure. Note that the embodiment of FIG. 2 adopts the reference numerals and part of the content of the embodiment of FIG. 1A to FIG. 1C, the same or similar reference numerals are used to represent the same or similar elements, and the description of the same technical content is omitted. For the description of the omitted part, refer to the foregoing embodiments, which is not be repeated herein.

Referring to FIG. 2, the difference between an ultrasonic transducer device 20 of FIG. 2 and the ultrasonic transducer device 10 of FIG. 1A is that the second opening OP2 of the second electrode 150 of the ultrasonic transducer device 20 is zigzag. In detail, the adjacent main parts 152 in the second direction D2 can be connected through the corresponding connecting parts 154, but the adjacent main parts 152 in the first direction D1 are not connected to each other. That is, the second electrode 150 is not a continuous structure and disconnected from each other in the first direction D1. Although in the embodiment, the second electrode 150 illustrated is discontinuous in the first direction D1, it is not intended to limit the disclosure. In other embodiments, the second electrode 150 may be discontinuous in the second direction D2 but continuous in the first direction D1.

In some embodiments, the first opening OP1 is rectangular, and the projection of the main part 152 on the insulating layer 120 is rectangular.

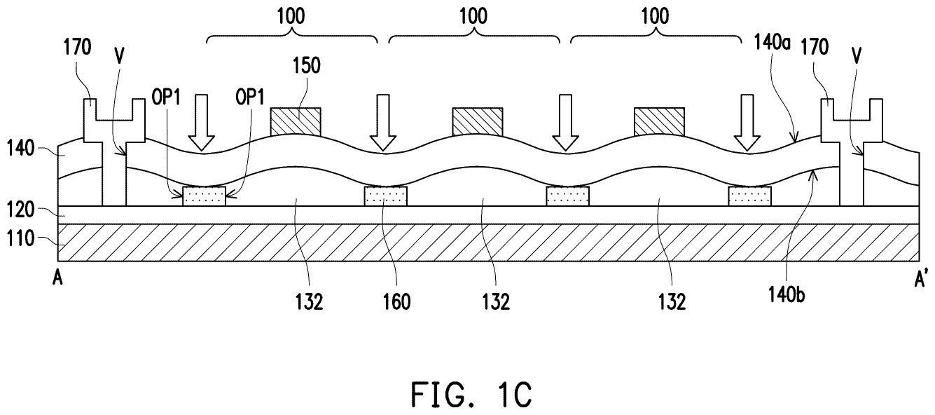

FIG. 3 is a schematic top view of an ultrasonic transducer device according to another embodiment of the disclosure. Note that the embodiment of FIG. 3 adopts the reference numerals and part of the content of the embodiment of FIG. 1A to FIG. 1C, the same or similar reference numerals are used to represent the same or similar elements, and the description of the same technical content is omitted. For the description of the omitted part, refer to the foregoing embodiments, which is not be repeated herein.

Referring to FIG. 3, the difference between an ultrasonic transducer device 30 of FIG. 3 and the ultrasonic transducer device 10 of FIG. 1A is that the second opening OP2 of the second electrode 150 of the ultrasonic transducer device 30 is rectangular.

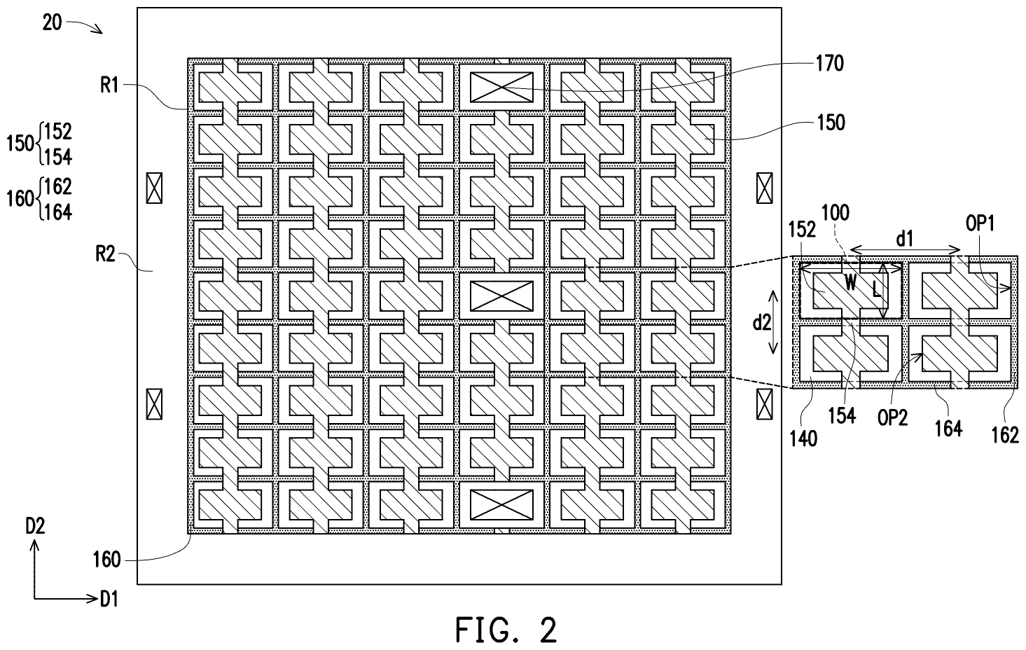

FIG. 4 is a schematic top view of an ultrasonic transducer device according to another embodiment of the disclosure. Note that the embodiment of FIG. 4 adopts the reference numerals and part of the content of the embodiment of FIG. 1A to FIG. 1C, the same or similar reference numerals are used to represent the same or similar elements, and the description of the same technical content is omitted. For the description of the omitted part, refer to the foregoing embodiments, which is not be repeated herein.

Referring to FIG. 4, the difference between an ultrasonic transducer device 40 of FIG. 4 and the ultrasonic transducer device 10 of FIG. 1A is that the second opening OP2 of the second electrode 150 of the ultrasonic transducer device 40 is round or oval.

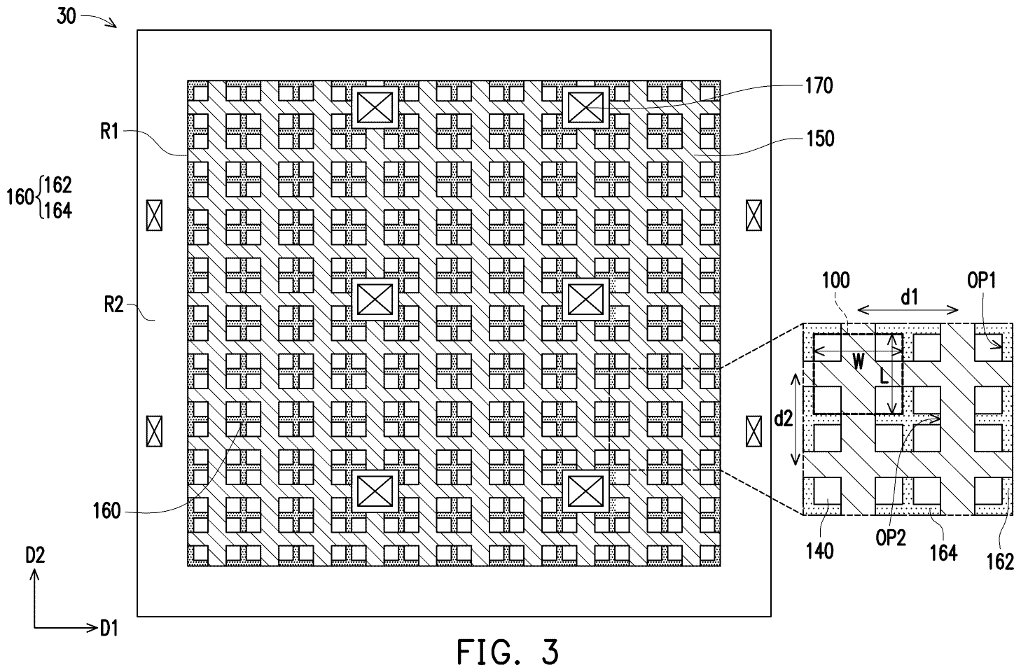

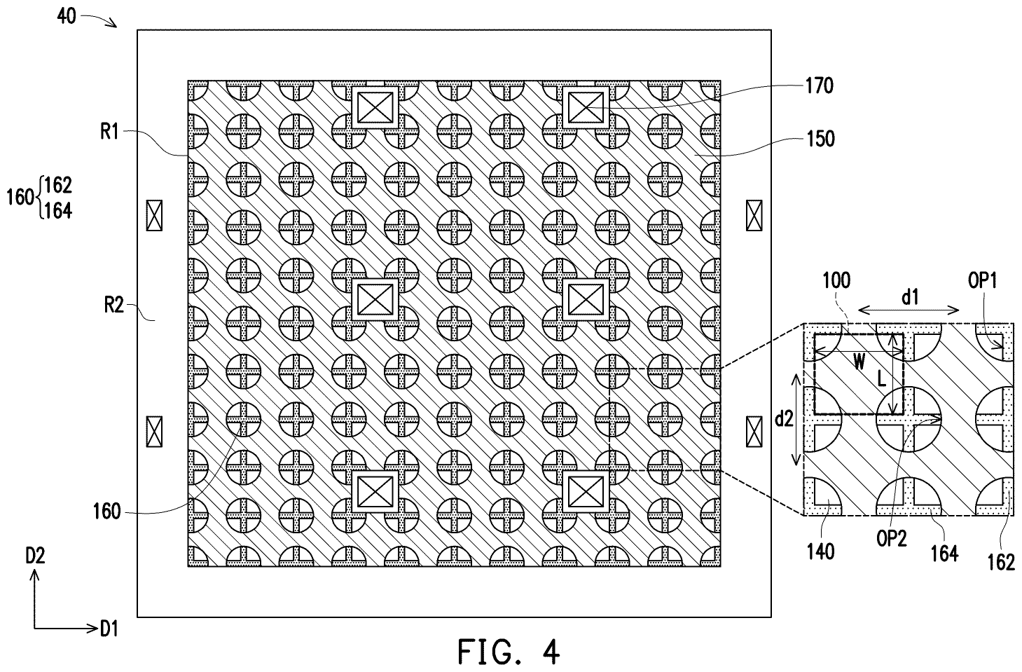

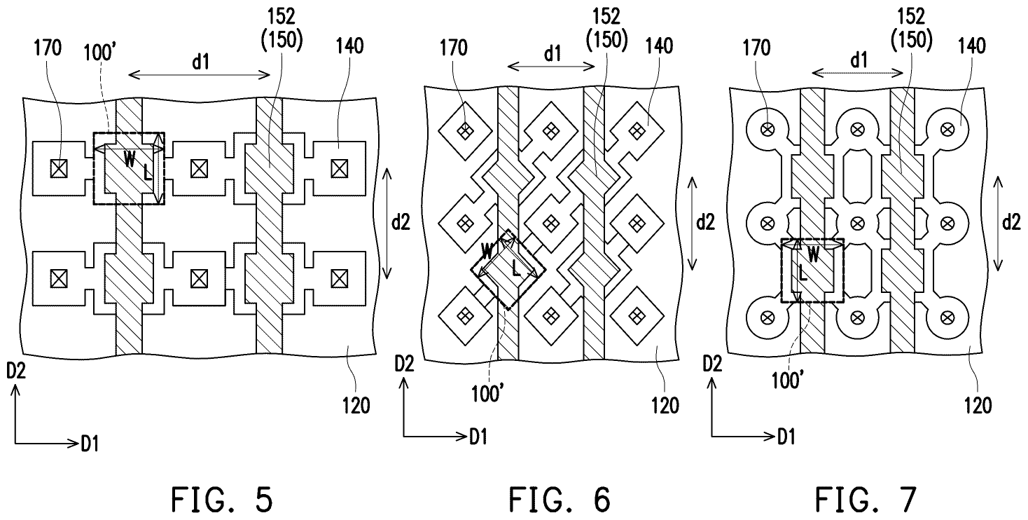

The following examples are given to verify the efficacy of the disclosure, but the disclosure is not limited to the followings. Note that the comparative examples of FIG. 5 to FIG. 7 adopt the reference numerals and part of the content of the embodiments of FIG. 1A to FIG. 1C, and the same or similar reference numerals are used to represent the same or similar elements, and the description of the same technical content is omitted. For the description of the omitted part, refer to the foregoing embodiments, which is not be repeated herein.

In the case in which the following Embodiments 1-2 and Comparative examples 1-3 have the same overall area, that is, a length of 300 μm and a width of 4500 μm, the differences in cell density of the ultrasonic transducer units resulting from various configuration of the ultrasonic transducer units are compared.

The ultrasonic transducer device of Embodiment 1 is similar to that of the embodiment of FIG. 1A to FIG. 1C, and the ultrasonic transducer device of Embodiment 2 is similar to that of the embodiment of FIG. 2. The ultrasonic transducer devices of Comparative Examples 1 to 3 all include the first electrode 110, the insulating layer 120, the oscillating membrane 140 and the second electrode 150 but with no third electrode. There is a cavity between the oscillating membrane 140 and the insulating layer 120, and there is the filling material 170 between adjacent ultrasonic transducer units 100′, but the configuration between ultrasonic transducer units 100′ and the filling material 170 of Comparative Examples 1 to 3 is different, as shown in FIG. 5 to FIG. 7, respectively.

The relative dimensions, numbers, area ratios and cell densities of the ultrasonic transducer units of Embodiments 1-2 and Comparative examples 1-3 are listed in Table 1. The dimensions of the ultrasonic transducer units of Comparative examples 1-3 of Table 1 refer to the width W*length L of the oscillating membrane 140 corresponding to the main part 152 of the second electrode 150. The distances d1 and d2 refer to the distance between the centers of two adjacent ultrasonic transducer units 100/100′ in the first direction D1 and the second direction D2. The area ratio refers to the ratio of the total area of the ultrasonic transducer unit to the overall area of the active region R1 of the ultrasonic transducer device. The cell density is used to calculate the ratio of the area of the ultrasonic transducer unit to the area of the through hole. For example, in Comparative examples 1-3, the number of ultrasonic transducer units is equal to the number of through holes, so the cell density is (the area of one ultrasonic transducer unit)/(the area of one ultrasonic transducer unit+the area of a through hole); for Embodiments 1-2, the number of ultrasonic transducer units is n times (e.g., 15 times) the number of through holes, so the cell density is (the area of n ultrasonic transducer units)/(the area of the n ultrasonic transducer units+the area of a through hole).

TABLE 1

| | dimensions of the | | | | |
|---|---|---|---|---|---|
| | ultrasonic | | The number of | | |
| | transducer unit | distance d1/ | the ultrasonic | area | Cell |
| the ultrasonic | (with W μm* | distance d2 | transducer | ratio | density |
| transducer device | length L μm) | (μm/μm) | units | (%) | (%) |
| Embodiment 1 | 20*20 | 23/23 | 2049 | 60.71 | 72.23 |
| Embodiment 2 | 40*20 | 43/23 | 1122 | 66.49 | 76.62 |
| Comparative | 20*20 | 40/40 | 784 | 23.23 | 25.00 |
| example 1 | | | | | |
| Comparative | 20*20 | 33.28/33.28 | 1072 | 31.76 | 36.12 |
| example 2 | | | | | |
| Comparative | 20*20 | 30.62/30.62 | 1314 | 38.93 | 42.66 |
| example 3 | | | | | |

The ultrasonic transducer units 100 of Embodiments 1-2 are formed by forming the sub-cavity 132 among the insulating layer 120, the oscillating membrane 140 and the third electrode 160 when the third electrode 160 is applied with a bias voltage, so more ultrasonic transducer units 100 can be configured in the same area, or the ratio of the area occupied by the ultrasonic transducer units 100 is relatively high, and the cell density is higher, thereby improving the bandwidth and the power output of the ultrasonic transducer device.

What is claimed is:

1. An ultrasonic transducer device, comprising:
a first electrode, a second electrode and a third electrode;
an oscillating membrane having a first surface and a second surface, disposed between the second electrode and the third electrode; and
an insulating layer having a top surface and a bottom surface, disposed on the first electrode,
wherein the third electrode is disposed between the oscillating membrane and the insulating layer,
wherein the insulating layer is located between the third electrode and the first electrode, and
wherein the third electrode is a mesh structure in direct contact with the insulating layer to form a plurality of sub-cavities defined by the mesh structure, the second surface of the oscillating membrane, and the top surface of the insulating layer.

2. The ultrasonic transducer device of claim 1, wherein a bias voltage is applied to the third electrode to generate a voltage difference between the third electrode and the first electrode.

3. The ultrasonic transducer device of claim 1, wherein the sub-cavities are closed spaces and are separated from each other.

4. The ultrasonic transducer device of claim 1, wherein the second electrode comprises a plurality of main parts and a plurality of connecting parts, wherein the main parts overlap the sub-cavities respectively, wherein each of the connecting parts is connected between corresponding two of the main parts, and wherein an area of each of the main parts is greater than that of each of the connecting parts.

5. The ultrasonic transducer device of claim 4, wherein the mesh structure includes a plurality of longitudinal portions arranged along a first direction and a plurality of transverse portions arranged along a second direction, wherein the first direction and the second direction intersect with each other, and wherein a plurality of first openings of the mesh structure are defined by the longitudinal portions and the transverse portions.

6. The ultrasonic transducer device of claim 5, wherein the mesh structure, the insulating layer, the oscillating membrane and the sub-cavities constitute a plurality of ultrasonic transducer units arranged in an array, wherein the ultrasonic transducer units correspond to the first openings of the mesh structure respectively, wherein a width of each of the ultrasonic transducer units is equal to that of each of the first openings of the mesh structure, and wherein a length of of each of the ultrasonic transducer units is equal to that of each of the first openings of the mesh structure.

7. The ultrasonic transducer device of claim 6, wherein the oscillating membrane is wavy, having a plurality of crests corresponding to the sub-cavities respectively, and a plurality of troughs corresponding to the longitudinal portions and the transverse portions of the third electrode.

8. The ultrasonic transducer device of claim 5, further comprising a plurality of filling materials, wherein a material of each of the filling materials comprises cured photoresist, silicon-containing nitride, or silicon-containing oxide, wherein the oscillating membrane has a plurality of through holes, wherein each of the filling materials contacts the first surface of the oscillating membrane and passes through the through holes of the oscillating membrane respectively to be in direct contact with the top surface of the insulating layer.

9. The ultrasonic transducer device of claim 8, wherein each of the filling materials does not contact the longitudinal portions and the transverse portions of the mesh structure.

10. The ultrasonic transducer device of claim 8, wherein the mesh structure, the insulating layer, the oscillating membrane and the sub-cavities constitute a plurality of ultrasonic transducer units arranged in an array, wherein the ultrasonic transducer units are located in an active region and are not in a peripheral region surrounding the active region, wherein the filling materials are distributed in the active region and the peripheral region.

11. The ultrasonic transducer device of claim 1, wherein the second electrode comprises a plurality of main parts and a plurality of connecting parts, wherein the main parts overlap the sub-cavities respectively, wherein each of the connecting parts is connected between corresponding two of the main parts in a first direction and is connected between corresponding two of the main parts in a second direction to constitute a plurality of second openings.

12. The ultrasonic transducer device of claim 11, wherein each of the second openings is cross-shaped, rectangular, round, or zigzag.

13. An ultrasonic transducer device, comprising:
a first electrode, a second electrode and a third electrode;
an oscillating membrane having a first surface and a second surface, disposed between the second electrode and the third electrode; and
an insulating layer having a top surface and a bottom surface, disposed on the first electrode,
wherein the third electrode is disposed between the oscillating membrane and the insulating layer,
wherein a cavity is formed between the oscillating membrane and the insulating layer, and
wherein the third electrode is a mesh structure disposed within the cavity, having a plurality of first openings.

14. The ultrasonic transducer device of claim 13, wherein the mesh structure contacts oscillating membrane and does not contact the insulating layer.

15. The ultrasonic transducer device of claim 13, wherein the second electrode comprises a plurality of main parts and a plurality of connecting parts, wherein each of the connecting parts is not connected between corresponding two of the main parts arranged in a first direction, and is connected between corresponding two of the main parts in a second direction, wherein the first direction and the second direction intersect with each other, and wherein the main parts of the second electrode overlap the first openings of the mesh structure respectively.

16. The ultrasonic transducer device of claim 13, wherein the second electrode comprises a plurality of main parts and a plurality of connecting parts, wherein each of the connecting parts is connected between corresponding two of the main parts arranged in a first direction and is connected between corresponding two of the main parts in a second direction to constitute a plurality of second openings.

17. The ultrasonic transducer device of claim 16, wherein each of the second openings is cross-shaped, rectangular, round, or zigzag.

18. The ultrasonic transducer device of claim 13, wherein a bias voltage is applied to the third electrode to generate a voltage difference between the third electrode and the first electrode such that the third electrode is brought close to the first electrode and contact the insulating layer to form a plurality of sub-cavities corresponding to the first openings of the mesh structure respectively, wherein the sub-cavities are defined by the mesh structure, the second surface of the oscillating membrane, and the top surface of the insulating layer, and wherein a size of each of the sub-cavities is smaller than that of the cavity.

19. The ultrasonic transducer device of claim 18, wherein the mesh structure includes a plurality of longitudinal portions arranged along a first direction and a plurality of transverse portions arranged along a second direction to define the first openings of the mesh structure, wherein the first direction and the second direction intersect with each other, and wherein the oscillating membrane is wavy, having a plurality of crests corresponding to the sub-cavities respectively, and a plurality of troughs corresponding to the longitudinal portions and the transverse portions of the third electrode.

20. The ultrasonic transducer device of claim 13, wherein the mesh structure includes a plurality of longitudinal portions arranged along a first direction and a plurality of transverse portions arranged along a second direction to define the first openings of the mesh structure, and wherein the first direction and the second direction intersect with each other.
